(19) United States
(12) Patent Application Publication
(10) Pub. No.: US 2010/0057095 A1
(43) Pub. Date: Mar. 4, 2010

(54) Method of Refraction Surgery of the Eye and a Tool for Implanting Intraocular Refractive Lens

(75) Inventors: Aslan Ramazanovich Khuray, Moscow (RU); Ilyas Amirovich Latypov, Moscow (RU)

Correspondence Address: PATENT, COPYRIGHT & TRADEMARK LAW GROUP, 4199 Kinross Lakes Parkway, Suite 275, RICHFIELD, OH, 44286, US

(73) Assignee: Leonid Orbachevsky

(21) Appl. No.: 12/614,643

(22) Filed: Nov. 9, 2009

Related U.S. Application Data
(63) Continuation-in-part of application No. 10/569,111, filed on Feb. 21, 2006.

(51) Int. Cl. A61F 9/013

(52) U.S. Cl. 606/107

(57) ABSTRACT

A reduction of eye trauma is achieved during opthalmolic surgery branch for implanting the intraocular refractive lens to the anterior chamber of the eye. The pupil is extended by mydriatic compounds and after anesthesia the cornea cut is made (clear cornea—3 mm). Thereafter, the anterior chamber of the eye is filled with viscoelastic compound with low molecular weight and then the refractive lens is implanted with the help of said cannula, the working face of the cannula at the middle between the lens edge and the border of the optic area, with the edge bent on the cannula face. The end by the top of the bending the refractive lens is introduced into the cornea cut and set in the posterior chamber of the eye. Thereafter, vacuum is removed and the cannula is detached from the refractive lens, and taken off the anterior chamber of the eye by the reverse movement. The refractive lens cannula is made as a tube with round or oval cross-section with inner diameter 0.5-2.5 mm and wall thickness not less than 0.05 mm. The tube is bent at 110-160, supplied with limiter, and working end that has diameter of the round cross-section of 1.0-2.0 mm or ellipse-shaped cross-section with small and big axes 0.6-0.9 mm and 1.5-2.5 mm, respectively.

### RELATED APPLICATIONS

The present invention in a Continuation in Part of U.S. Ser. No. 10/569,111, filed on Feb. 21, 2006, which was a National Phase application of PCT/RU2004/000324 having an International filing date of Aug. 18, 2004 and an International priority date of Aug. 21, 2003.

### BACKGROUND OF THE INVENTION

1. Field of the Invention

The present invention relates generally to opthalmologic surgery and, more particularly, to a opthalmologic operations for implanting intraocular refractive lens to the anterior chamber of the eye, and a tool forming a canulla for fixing a refractive phakic by vacuum to allow implantation of posterior-chamber phakic intraoccular lenses to the posterior chamber of the eye.

2. Description of the Related Art

Implantation of posterior-chamber phakic intraoccular lenses (referred to also as PIOL) for correction of ametropies becomes more and more popular. However, the lack of complexity of the surgical technologies for current use in such implantation makes it difficult for applying by many opthalmosurgeons.

Refraction surgery requires ultra-compact and accurate sizes, which would allow for operating in a strictly limited space, such as, for example, the anterior chamber of the eye, where the depth (about 3 mm) and slight movements cause irreversible and dreadful consequences for the crystalline lens and cornea endothelium.

A search of the prior art did not disclose any patents that read directly on the claims of the instant invention; however, references considered related were described in the parent applications of the present invention, which are incorporated by reference herein as if rewritten in their entirety.

Additional references are also thought to be relevant.

U.S. Pat. No. 6,273,894, issued in the name of Dykes, discloses an apparatus for positively gripping and manipulating an implant within the eye that includes a vacuum cannula apparatus. Such device is meant for an entirely different purposes, namely to insert the haptic elements of the lens which has been already implanted from the anterior chamber of the eye into the posterior chamber of the eye and it has different design. With the help of the subject instrument it is impossible to implant the refractive phakic lens into the anterior chamber of the eye because its design will cause the rupture of the lens and will damage the eye tissues (iris, crystalline lens, cornea).

U.S. Pat. No. 5,052,999, issued in the name of Klein, discloses a liposuction apparatus that incorporates a vacuum control within an improved handle for regulating the amount of suction applied during a liposuction procedure.

U.S. Pat. No. 4,631,935, issued in the name of Berryessa, discloses a cardioplegia/air aspiration cannula assembly for delivering cardioplegic fluid to and for aspirating air from the aorta during surgery.

U.S. PGPub No. 2004/0054373A1, published in the name of Weber et al., discloses an apparatus and method for delivering ocular implants or microimplants which forms essentially an ergonomically design syringe for the injection of implants or microimplants.

U.S. Pat. No. 4,674,502, issued in the name of Imonti, discloses an intra ocular surgical instrument having an elongate dinner tube that is axially movable within an outer tube that is connected to a handpiece. A cutting edge is formed between the two tubes.

And, U.S. Pat. No. 6,090,121, issued in the name of Weber et al., disclosed a swan neck liposuction cannula having a long, flexible plastic shaft capable of being bent into a semi-circle without breaking and yet capable of returning to its original shape.

While various elements within these references may possibly teach features capable of being adapted to a cannula-shaped working component in combination with a vacuum source, they do not teach a design for a tool intended for installation of phakic lenses in an eye. The technical task, to solve which a number of inventions is aimed, is decrease of the eye tissue traumatism in the operation zone and improvement of the operation quality. This target has been reached by long-term work experimentation with the design of the tool (cannula) and components of the tool (a hoof, a corner of an inclination of a hoof, a pusher, the area of contact with the lens, vacuum, diameter of the cannula). Currently, there are only two tools that allow for the establishment of phakic lenses in an eye: an injector; and a tweezers. Clinical use of the cannula of the present design through experimentation and use in Russia has shown that such design of the offered tool allows to reach our target, preventing possible complications and reducing operation time in comparison with an injector and a tweezers, a design and which technics of implantation considerably differs from suggesting tool.

While the opthalmologic tools of the related art allow the surgeon to make various manipulations in an eye (such as cataract removal, crystalline lens destruction, lens positioning in an eye). In their design, certainly, there could be involved such elements as the described and anticiapted does not allow to establish phakic lens in an eye.

Of additional relevance is U.S. PGPub No. 2003/0216747A1, published in the name of Kaplan. While the Kaplan reference is not ‘prior’ art in that the publication date of Kaplan is Nov. 20, 2003 and the priority date of the present application is Aug. 21, 2003 (prior to the date that Kaplan became available as a reference), this reference does show the subtlety in distinguishing features and characteristics when intended to function as a Tool for Implanting an Intraocular Refractive Lens as in the present invention. By example, Kaplan teaches away from the present invention in that it is designed, intended and teaches expression of retinal graft material “from the opening into the subretinal space of the recipient eye”. Kaplan further incorporates a “plunger . . . inserted through the plunger opening into the plunger tavel channel to express fluid in the reservoir” is driven by an electronmechanical drive. The delivery opening is designed to “reach sufficiently to be epiretinally located or to reach into the subretinal space of a mammalian or human eye”. Further still, the “bend at the distal end of the cannula 28 can be eliminated in some instances. The tip 30 can be bent to accommodate the shape of the BACK of the eye”.

Generally, the Kaplan reference, as with all the art prior to the present invention, can accommodate removal or destruction of lens material, but are in no way capable of the delicate manipulation required to move or place an intraocular lens in a sufficient manner without injury or destructions of the tissue.

Consequently, a need has been felt for providing an apparatus and method to solve which a number of inventions is aimed, is decrease of the eye tissue traumatism in the operation zone and improvement of the operation quality.

### SUMMARY OF THE INVENTION

It is therefore an object of the present invention to provide an improved method of refraction surgery of the eye and a tool for implanting intraocular refractive lens.

The present invention includes a number of features that are unanticipated in the above mentioned references, including, but not limited to:
a linear elongated, hollow tube having a smooth cross section and bent at an angle such as to terminate at a contact end for supporting a surface of the lens;
a handle connected to said working component and having a working end opposite a vacuum end,
said handle forming a hollow conduit in fluid communication with said working component;
a source of vacuum in fluid communication with said working component through said conduit in said handle; and
a control means for controlling and limiting said source of vacuum at a sufficient level such as to fixe said lens to said contact end.

Briefly described according to one embodiment of the present invention, this technical problem was solved by a method that according to the current invention, including cornea cutting, fixing and implantation of said refractive lens by said operating tool, after anesthesia and extending the pupil by mydriatic compounds cornea is cut (to clear the cornea by 3 mm). After that the anterior chamber is filled by a viscoelastic compound of low molecular weight and said refractive lens is implanted using the cannula connected to 40-60 mmHg vacuum source. For this purpose, said refractive lens is fixed by the working face of the cannula at the middle between the lens edge and the optic are border, when the edge is bent to the cannula face. Then the top of the lens bend is injected into the cornea cut, and the lens is set in the posterior chamber of the eye. When vacuum is eliminated, the cannula is detached from said refractive lens, and said cannula is removed from the anterior chamber.

Said technical task was also resolved by the composition of the tool for said refractive lens implantation, which, according to said invention, has the operating part for fixing and carrying said refractive lens through the cornea cut to the anterior chamber of the eye and the handle. In said tool, the operating part is shaped as a cannula representing a tube bent at the angle of 120°-130°, extending the oval-shaped cross-section behind the bending and trumpet-shaped working face, 0.4-0.6 mm long and ellipse-shaped cross-section with the minor and major axes equal 0.8-0.9 and 1.8-2.0 mm, respectively.

As anticipated by the present invention, the cannula is made from biologically inert material with the tensile strength 27 MPa or higher. The edge and the internal surface of said trumpet-shaped working face of the cannula are covered a polymer with tensile strength 2.9 3 MPa. Said handle for cannula manipulation represents an evacuating syringe, connected to the cannula via a socket.

Moreover, said cannula is made from LDPE and stainless steel, and said covering material is polyurethane methacrylate.

An advantage of the present invention is that the implantable PIOL is not mechanically fixed, avoiding the lens damage danger;

Another advantage of the present invention is that the rotation of PIOL inside the cartridge and respective improper installation are impossible.

Yet another advantage for the present invention includes the level of traumatism of both implanted PIOL and the surrounding eye tissues are reduced.

Yet another advantage of the resent invention, when compared with other methods, is that operation time is reduced, because implantation is performed by a single move.

Further, the simplicity of the method allows familiarization of this operation in the high opthalmosurgeonshipm, while unitizing of the tool handle with vacuum syringe makes the tool compact and comfortable for the surgeon hand.

Further still, the tool of the present invention suggested can be provided in an inexpensive and disposable device, thereby eliminating sterilization and reduces hazard of patient's infection.

### BRIEF DESCRIPTION OF THE DRAWINGS

The advantages and features of the present invention will become better understood with reference to the following more detailed description and claims taken in conjunction with the accompanying drawings, in which like elements are identified with like symbols, and in which:

FIG. 1 is shows the general scheme of operation of the preferred embodiment of the present invention;

FIG. 2 demonstrates the general overview of the device;

FIG. 3 is a partial side view thereof;

FIG. 4 is a cross sectional view thereof, taken alone line a-a of FIG. 3;

FIG. 5 is a cross sectional shape of the tube with round cross section;

FIG. 6 is a cross sectional shape of the tube with oval-shaped cross section;

FIG. 7 shows the connection of the cannula with handle and the source of vacuum; and

FIG. 8 demonstrates an alternate method of supply the cannula with vacuum.

### DESCRIPTION OF THE PREFERRED EMBODIMENTS

The best mode for carrying out the invention is presented in terms of its preferred embodiment, herein depicted within the Figures.

### 1. Detailed Description of the Figures.

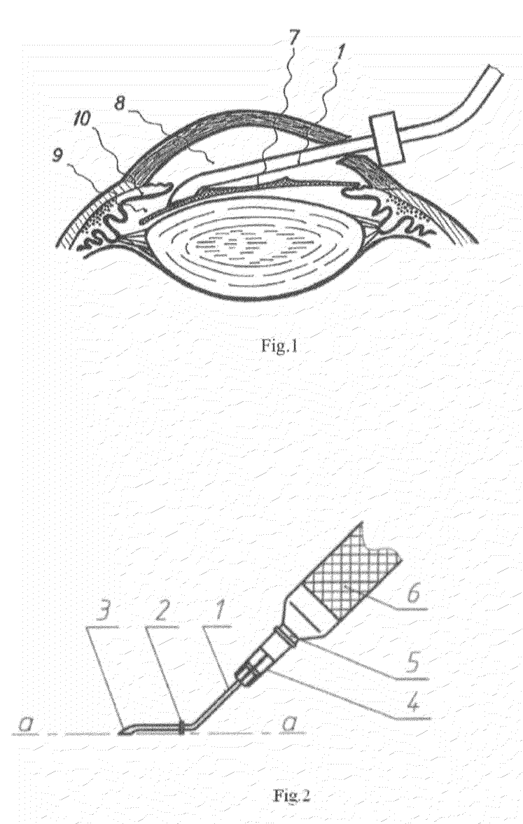

FIG. 1 shows the overall scheme of operation of the present invention. In conjunction with FIG. 2, the general overview of the device is demonstrated and shows the cannula shaped as a bent tube at an angle of between 110-160. Equipped with a limiter 2, the cannula has a V-shaped working end or face 3 when viewed from the conditional horizontal surface “a-a:. The Cannula is also supplied with a socket 4, established on the air outlet 5 of the handle 6 connected with the vacuum.

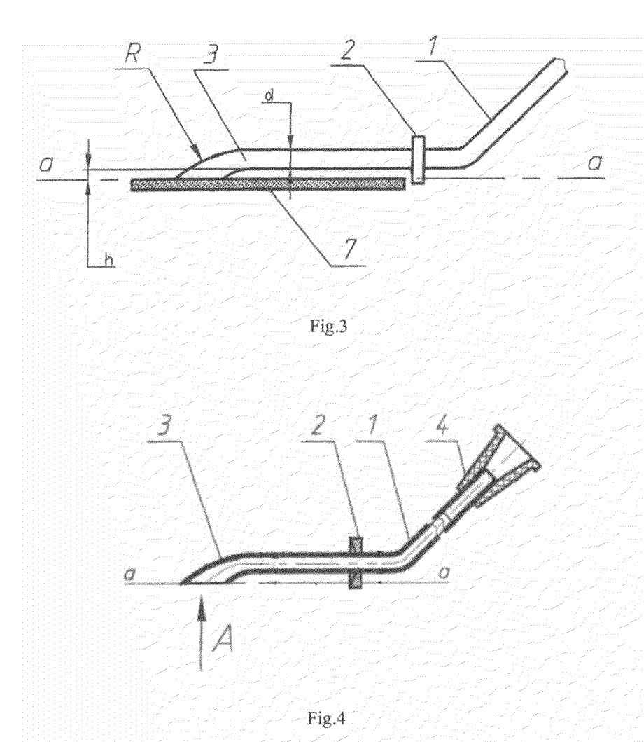

FIG. 3 shows the bend of the working end 3 fixed on the haptic part of the lens 7, at the same time the radius of the bend of the working end is not less than 2 diameters of the tube.

FIG. 4 demonstrates the design of the cannula; the limiter 2, established at the length 7-14 mm from the working end 3, is made with conical funnel 100-150, which fixates and moves the intraocular lens, and also limits the excessive movement of the lens into the anterior chamber of the eye.

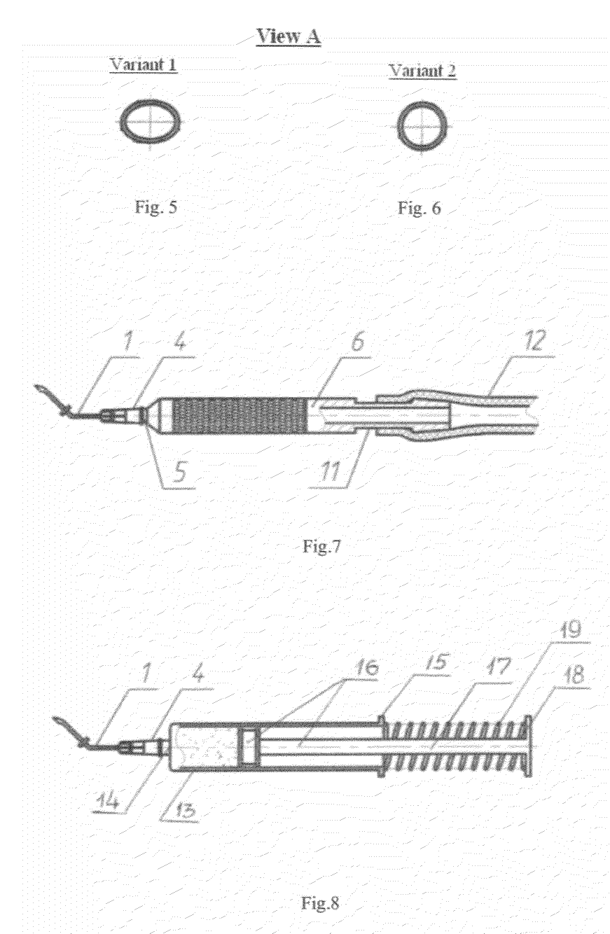

FIG. 5 and FIG. 6 show the cannula made in the shape of a tube, with round or oval-shaped cross section, respectively, with an inner diameter of 0.5-2.5 mm and the thickness of the wall not less than 0.05 mm. The working end 3 has a round cross-section with the diameter 1.0-2.0 mm or ellipse-shaped cross-section with small and big axes 0.6-0.9 mm and 1.5-2.5 mm, respectively.

FIG. 7 shows the connection of the cannula 1 with socket 4 with air outlet 5, handle 6, that has rough surface (notches). The handle is made hollow and by air outlet 11 connected with the flexible trumpet 12 and with the source of vacuum, for example phacoemulsificator “Millennium”.

FIG. 8 shows an alternate variant for vacuum for the cannula. The handle in the shape of a cylinder 13, made from transparent material, can be used as a source of vacuum. IT has air outlet 14 for connecting with cannula 1 on one end and crimp seal 15 on the other end. Inside the cylinder there is a spring-loaded piston 16 with rod 17, supplied with platter 18, commensurable by the area of the surface with crimp seal. At the same time, compression spring 19, made of steel wire, is selected with parameters to establish a vacuum of 100-600 mmHg. The syringe with spring rod can serve as a source of the vacuum, the characteristics of the spring provide specified vacuum. For example, in the cylinder with diameter 10 mm, length 50 mm and piston stroke of 40 mm, at compression of spring with 450 g and advancement of the piston toward air outlet connection with cannula, the portion of air is pressed out. At the application of the cannula end to the lens and release of the piston, the compression spring is released, it turn removes piston inside the cylinder from air outlet and cannula, and creates the vacuum of 325 mmHg (0.427 atm.); the lense is securely fixed. After placement of the intraocular refractive lens into posterior cavity of the eye with the pressing of the piston, the remaining portion of the air pushes the lens out of the cannula. During this process, the cylinder serves as a handle and the transparent wall of the cylinder allows to have visual control of the tool's performance. In this example, the steel wire was selected with the diameter 1 mm, the coiling diameter 10 mm and 3 mm step; and at the same time the spring is fixed and treated at the temperature of 400. The dependence of the required vacuum can affect the parameters of the spring and will allow use of different types and sized of cylinder and spring.

Said lens is fixed to said cannula face due to vacuum (40-60 mmHg deep) that excludes any mechanical damaging of said lens. Said cannula is of miniature size, determined experimentally. Various shapes of said cannula working face have been tested, for example, a polyethylene tube reinforced by a stainless steel tube, with cylindrical or oval-shaped soft working face.

Experimentally, it has been found that LDPE mechanical parameters are satisfactory for preserving the bent shape of the cannula tube and the strength of the whole structure, required during the operation. This material trademark has been selected among analogous polymeric materials, such as poly(methyl methacrylate) (PMMA) collagen, hydrogel. Said material has the following parameters: tensile at break 400 MPa, tensile strength 27 MPa, elasticity modulus at bending 550-700 MPa. As said tool and technique of said intraocular refractive lens implantation was developed, the cannula made from stainless steel showed equal results. Meanwhile, it has been found that said working face must be shaped as a hollow paddle (a flattened funnel) with a hole enough for said lens suction by vacuum and the external surface, which causes no damage to the lens bending at its advancing. The material selected for the working face is polyurethane methacrylate as tensile strong as 2.9 3.0 MPa, covering the edge and the internal surface of the V-shaped face of said cannula.

Geometrical sizes were selected experimentally with respect to the size of the operation cut and the lens to be implanted. Said tool has no mechanisms, which would be moved inside the eye. Detachment of said cannula from said intraocular refractive lens requires no manipulations in the anterior chamber of the eye. Smooth and straight surface of said cannula eliminates the danger of damaging tissues during dragging the tool out from the eye. At the implantation, there are no contacts of said cannula and the eye tissues, and any contact between said cannula and the front surface of the crystal capsule is eliminated. Since said cannula is moved in parallel to the crystal of the eye and there is a limiter behind said cannula bending, any crystal semiluxation is eliminated. Said cannula body trespassing through the cornea cut prevents bleeding of the anterior chamber of the eye that allows for preserving the anterior chamber of the eye operating. Said lens turning upside down id eliminated, because said lens is fixed. Said lens can be visually monitored during the operation. Any opportunity of unwilling re-implanting of said lens during reverse movement of said cannula is eliminated. Said tool is inexpensive one-shot tool that excludes sterilization procedures and reduces danger of personal infecting of the current patient. The unique handle of the tool with said evacuating syringe makes the tool rather compact and suitable for a surgeon and his hand.

The technique suggested cut time of operation and avoids any complications, because the operating tool fixes said lens so that the cannula is enveloped in the lens, and any contact with the anterior capsule of the crystal and cornea endothelium is completely avoided.

### 2. Operation of the Preferred Embodiment

The performance of the tool is encompassed in fixating the lens by the working end of the cannula using vacuum, the advancement of the lens into the anterior, then into the posterior cavity of the eye and implantation of the haptic part of the lens under the iris of the eye. After removing the vacuum the lens is detached from the working end of the cannula and the cannula is removed from the surgery field.

According to said invention, the method for refraction surgery of the eye, the intra ocular refractive lens implantation is implemented as follows. After local anesthesia 3 mm long cut of cornea with preliminary medicated medryase is made. Thereafter, the anterior chamber is filled with a viscoelastic compound of low molecular weight and said intraocular refractive lens is implanted using said cannula, evacuated to 40-60 mmHg. Said lens is fixed by the trumpet-shaped face in the middle between the lens edge and the border of the optical area. The edge of said lens is bent on the cannula face and then by the bend top the lens is introduced into the cornea cut. Thereafter, said lens is fixed in the anterior chamber of the eye, vacuum is removed and the cannula is detached from said intraocular refractive lens, and said cannula is removed from the anterior chamber.

The existing complexity of the surgical operation at implantation of the intraocular refractive lens limits the possibility of its wide application to the opthalmologic practice. Said invention allows for simplifying the operation technique and ring-fence both a patient and a surgeon from possible complications. The general impression is that the patent tool is extremely simple and has nothing new in its structure. It seems that its design is obvious and can be implemented by any specialist acquainted with the problems of refractive surgery.

However, while fixation of parts using vacuum is well known (for example, U.S. PGPub No. 2003/0216747A1, published in the name of Kaplan as indicated above), in ophthalmology, damaged lens is removed using vacuum. Theoretically, using a tube and vacuum a thin lens may be held; however, implantation may not be performed by such a tool. The particular adaptations and teachings of the present invention can solve multiple tasks to make is use functional for the intended purpose. These include:
1. Prior to installation, at the beginning of the operation the lens shall by simply and reliably fixed;
2. All displacements of the lens by the tool ace controlled by a surgeon;
3. The tool does not damage the lens and tissues of the eye; and
4. The lens with the tool shall pass into the chamber of the eye through a minimal cut.
   Here problems occur. The cut size is much smaller than the lens cross-section. When passing the cut, the lens “wings” shall bend and lie on the tool; hence, both the lens and the fixation zone sustain significant mechanical loads. Lens damage, eye tissue traumatizing, and lens detachment are inadmissible.

Additional task include:
5. The operation time shall be as short as possible;
6. After penetration into the chamber the lens shall be set to the place and laid down; and
7. When the tool is inserted the lens shall not be displaced and eye tissues damaged.

Taking into account a complicated configuration of the objects, a definite elasticity of the eye tissues, some range of sizes and configurations of eyes, variable flexibility of the lens regarding thickness (diopter) and material, it is very difficult to select correct geometry of the tool, fixation site size and holding effort. Selection of optimal tool geometry, especially the contact site and bending value, has taken approximately three years and has included the making and testing of over 50 pilot models in an iterative fashion. Each model was first tested on cadaveric eyes, and then on rabbits.

Said models were tested by vacuum sucking on by said cannula in different sites. Several said tests performed at 30 mmHg vacuum indicate significant lens cut-in or sucking in of its surface by the cannula. For 70 mmHg vacuum, exfoliation of said lens surface from the cannula face was observed at its movement. Thus, 40-60 mmHg vacuum is optimal.

The experiments with imprints of said cannula face on said lens surface allowed for selecting optimal sizes of said cannula face flattened as the oval. A combination of cannula strength (avoiding its twisting and bending), the channel size, lens preservation, easiness of penetration through the cornea cut and taking it out of the tunnel.

The evacuation syringe connected to said cannula was tested for a possibility of making 40-60 mmHg vacuum. A cavity before the piston was connected to a pressure-rarefaction sensor and a limiter, a fixer for the piston movement was selected in order to make the structure and functioning of the vacuum syringe providing a surgeon reliability of all parameters.

Surgical operations were performed in the Moscow Research Opthalmologic Center “New Sight”. The clinical example: patient M (female), 32 years old, entered into the hospital with a diagnosis: high myopia OU, anisometropia, OD sph. −13.75=cyl. −2.5 ax 180. OS sph. −0.5=cyl. −1.5 ax 180, to whom LASIK of the left eye was performed on Sep. 7, 2003. The next operation on the right eye intraocular refractive lens implantation was performed on Sep. 14, 2003.

In the operation, the pupil is first medicamentally extended. After local anesthesia the cornea is 12 h direction cut by 3 mm. Said viscoelastic compound with low molecular mass is injected into the anterior chamber of the eye. Using efacuation, said intraocular refractive lens is fixed to the cannula face. Using said cannula said lens is implanted to the anterior chamber of the eye, then vacuum is removed, cannula is removed from said chamber by the reverse motion. Using an applicator said haptic of the lens is set properly under the iris of the eye. The anterior chamber of the eye is rinsed with a physiological solution. A pupil-narrowing compound (myocol) is injected to the anterior chamber and, using the pincer and micro-iris scissors, peripheral iridoectomy is performed. At the site of the cornea cut BSS solution for non-suture adaptation of the wound edges is injected to the stroma. The conjunctival sac is dropped by an antibiotic solution.

The next day after the operation the eye is still, cornea is transparent, and the anterior chamber contains clear fluid. The pupil actively reacts on the light influence, no contact with the front surface of the crystal is observed; the fundus of the eye shows pink reflux. The refraction equals OD sph. −0.75=cyl. −1.5 ax 180. Thus, 13.00 diopters were obtained.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the Claims appended hereto and their equivalents. Therefore, the scope of the invention is to be limited only by the following claims.

### 2. Operation of the Preferred Embodiment

In accordance with a preferred embodiment of the present invention, the technical result of realization of said method and the tool is as follows:
said intraocular refractive lens to be implanted is not mechanically fixed (compressed) by pincer faces, which eliminates danger of the lens damaging;
said intraocular refractive lens cannot be turned upside down inside the cartridge, whence improper implantation is eliminated;
a possibility of traumas for both intraocular refractive lens to implanted and the surrounding tissues of the eye is reduced;
the operation time is reduced compared with other methods, because implantation is complete in a single slight movement;
said method simplicity allows for adapting this operation to the broadest range of ophthalmologists;
retrieval of said tool handle is not expensive and should be made once.
   This excludes any sterilization, and danger of the patient infecting is reduced.

As compared with the prototype, it has been found that said method differs by the vacuum fixing of the lens (40 60 mmHg), bending of the lens edge and using it in the lens advancing by said tunnel until said lens is properly located and its surface is disconnected from said cannula face after removal of the vacuum. Said tool has an original construction of the operating part, also original parameters of said cannula material and sizes, and the moving handle implemented as a vacuum syringe.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the Claims appended hereto and their equivalents. Therefore, the scope of the invention is to be limited only by the following claims.

What is claimed is:

1. A tool for implantation of the refractive lens comprising:
a working component consisting of a cannula formed of a biologically inert material, said working component for fixing and introducing the refractive lens through the cornea cut into the cavity of the eye, wherein said working component forms a linear elongated, hollow tube having a smooth cross section and bent at an angle such as to terminate at a contact end for supporting a surface of the lens;
a handle connected to said working component and having a working end opposite a vacuum end, said handle forming a hollow conduit in fluid communication with said working component;
a source of vacuum in fluid communication with said working component through said conduit in said handle; and
control means for controlling and limiting said source of vacuum at a sufficient level such as to fixe said lens to said contact end.

2. The tool of claim 1, wherein said material is selected from the group comprising: surgical steel, titanium, and HDPE.

3. The tool of claim 1, wherein said smooth cross section is selected from the group comprising: round; elliptical; and oval.

4. The tool of claim 6, wherein said source of vacuum can be any system that provides necessary vacuum for the purpose of fixing said lens between 100-600 mmHg.

5. The tool of claim 1, wherein said handle connected with said source of vacuum is made of transparent material.

6. The tool of claim 1, wherein said control means for controlling and limiting said source of vacuum comprises:
a crimp seal formed at said vacuum end of said handle; and
a spring loaded piston with rod housed within said conduit in said handle having a spring for urging said piston against said crimp seal.

7. The tool of claim 1, further comprising:
a lubricant coating accumulated at said contact end for providing sealing and lubrication between said contact end and said lens.

8. A tool for implantation of the refractive lens comprising:
a biologically inert cannula-shaped working component for fixing and introducing the refractive lens through the cornea cut into the chamber of the eye, wherein said working component forms a linear elongated, hollow tube having a smooth cross section and bent at an obtuse angle such as to terminate at a contact end for supporting a surface of the lens without damage to said lens;
said contact end said working end forming an oval-shaped with minor axis between 0.6-0.9 mm and major axis between 1.5-2.5 mm;
a handle connected to said working component and having a working end opposite a vacuum end, said handle being laterally offset from said working component and forming a hollow conduit in fluid communication with said working component;
a source of vacuum in fluid communication with said working component through said conduit in said handle; and
control means for controlling and limiting said source of vacuum at a sufficient level such as to fixe said lens to said contact end.

9. The tool of claim 8, wherein said material is selected from the group comprising: surgical steel, titanium, and HDPE.

10. The tool of claim 9, wherein said smooth cross section is selected from the group comprising: round; elliptical; and oval.

11. The tool of claim 8, wherein said source of vacuum can be any system that provides necessary vacuum for the purpose of fixing said lens between 100-600 mmHg.

12. The tool of claim 8, wherein said handle connected with said source of vacuum is made of transparent material.

13. The tool of claim 8, wherein said control means for controlling and limiting said source of vacuum comprises:
a crimp seal formed at said vacuum end of said handle; and
a spring loaded piston with rod housed within said conduit in said handle having a spring for urging said piston against said crimp seal.

14. The tool of claim 8, further comprising:
a lubricant coating accumulated at said contact end for providing sealing and lubrication between said contact end and said lens. A tool for implantation of the refractive lens comprising:
a cannula-shaped working component for fixing and introducing the refractive lens through the cornea cut into the chamber of the eye;
a handle affixed to and extending from said cannula-shaped working component and forming a tube bent—with a concave limiter 8 mm in diameter and 3 mm wide after the bending, round or oval section;
said working end which is oval-shaped with minor and major axes between 0.6-0.9 mm and 1.5-2.5 mm, respectively;
said cannula made from a biologically inert material with tensile strength 27 MPa or higher;
said handle shaped as a hollow tube covered by a cellular knurl and adapted for moving said cannula; and
connection to a vacuum source via an elastic means connected to said cannula via a trumpet.

15. The tool of claim 14, wherein the said cannula is shaped as a tube made from low pressure polyethylene (LDPE), reinforced by a tube from stainless steel.

16. A method for implanting the intraocular refractive lens, said method utilizing a tool for implantation of the refractive lens of claim 8 and further comprising the steps:
a. extending the pupil of the eye by midriatic agents;
b. Cutting the cornea after anesthesia;
c. Filing the anterior cavity of the eye with means for retaining the hydrodynamic shape of the eye;
d. Implantation of the refractive lens into the cavity of the eye with the help of said cannula, that is connected to a source of vacuum, that fixates the refractive lens, wherein the contact end of cannula is placed on an upper surface of the haptic part of the lens, close to the border of the optic part, and fixed by 10-6000 mmHg vacuum; and
e. Advancing the cannula in the horizontal plane above the surface of the lens consecutively into said cavity of the eye.

17. The method of claim 16, wherein means for retaining the hydrodynamic shape of the eye comprises filling the cavity of the eye with viscoelastic compound.
